(19) United States
(12) Patent Application Publication
(10) Pub. No.: US 2014/0044690 A1
(43) Pub. Date: Feb. 13, 2014

(54) High-Energy Compounds for Use in Alzheimer's and Other Neurodegenerative Diseases

(71) Applicants: Ming CHEN, Seminole, FL (US); Darrell R. Sawmiller, Land O Lakes, FL (US); Huey T. Nguyen, Seminole, FL (US)

(72) Inventors: Ming CHEN, Seminole, FL (US); Darrell R. Sawmiller, Land O Lakes, FL (US); Huey T. Nguyen, Seminole, FL (US)

(73) Assignees: University Of South Florida; U.S. Department of Veterans Affairs, Washington, DC (US)

(21) Appl. No.: 13/961,820

(22) Filed: Aug. 7, 2013

Related U.S. Application Data
(60) Provisional application No. 61/742,325, filed on Aug. 8, 2012.

(51) Int. Cl. A61K 31/708; A61K 31/664; A61K 31/661; A61K 31/7076

(52) U.S. Cl. CPC A61K 31/708; A61K 31/7076; A61K 31/664; A61K 31/661
USPC 424/94.1; 514/47; 514/48; 514/118; 514/120; 435/375; 435/371

(57) ABSTRACT

A method of treating or preventing a neurodegenerative disease includes administering an effective amount of a compound to a subject in need thereof. The compound includes a high-energy compound, such as adenosine triphosphate (ATP), guanosine triphosphate (GTP), phosphocreatine (PCr), acetyl coenzyme A (ACoA), phosphoenol pyruvate (PEP), or a combination thereof.

### CROSS-REFERENCE TO RELATED APPLICATIONS

The present application claims priority on prior U.S. Provisional Application Ser. No. 61/742,325, filed Aug. 8, 2012, which is hereby incorporated herein in its entirety by reference.

### STATEMENT REGARDING FEDERALLY SPONSORED RESEARCH OR DEVELOPMENT

The work leading to the present invention was made with Government support, and specifically by the Merit Review program of the U.S. Department of Veterans Affairs. The U.S. Government, therefore, has certain rights in the invention.

### FIELD AND BACKGROUND OF THE INVENTION

The present invention is generally directed to drugs and therapies, and more particularly to the identification and discovery of high-energy compounds for use in the treatment or prevention of various neurodegenerative diseases.

A prominent feature of brain aging and Alzheimer's disease (AD) is the deposition of Aβ, a proteolytic fragment of amyloid-β precursor protein (APP). Physiological or α-processing of APP prevents the formation of Aβ (Esch et al. 1990; Walsh and Selkoe, 2004), but this pathway is somehow inactivated during aging with a concomitant increase of Aβ, reflecting a proteostasis failure (Lannfelt et al. 1995; Kern et al. 2006). It has been found that activation of APP α-processing inevitably decreases Aβ, and vice versa (Skovronsky et al. 2000; Frautschy et al. 1998; Etcheberrigaray et al. 2004). As such drugs promoting APP α-processing would potentially benefit the aging brain. Along this line, a range of compounds have been found to exhibit such effects including growth factors, hormones, cytokines, and stimulators of neurotransmitter receptors and signaling transduction pathways. These compounds have been under intense studies to evaluate their therapeutic values (Lichtenthaler, 2011; Sun and Alkon, 2010).

In our laboratory, we also tested a number of substances for their potencies to promote APP α-processing. These substances include, among others, current and experimental medications, herbal extracts and nutritional supplements. The search has led us to unexpectedly find that a group of high-energy compounds (HECs) is highly effective in promoting APP α-processing. Subsequent experiments showed that these HECs also boosted the survival of cultured neuronal cells impaired by energy inhibitors or oxidative stress.

HECs contain an energy-rich bond of ˜either phosphoryl or ˜acyl group, which upon hydrolysis liberates at least 7 kilocalories per mole of free energy (ΔG°) under standard conditions. There are many HECs in the body, the best known of which are adenosine, guanosine or cytidine triphosphate (ATP, GTP and CTP), phosphocreatine (PCr), acetyl coenzyme A (ACoA), phosphoenol pyruvate (PEP) and S-adenosylmethionine (SAM) (Lehninger, 1975; Nicholls and Ferguson, 2002). In central nervous system (CNS), free energy is the essential driving force for synthesis of macromolecules, fast axonal and dendritic transport and maintenance of ion gradients (McDaniel et al. 2003; Bonda et al. 2009).

### Aspects of the Invention

The present disclosure is directed to various aspects of the present invention.

One aspect of the present invention is to identify a high-energy compound(s) (HEC) that can enhance the energy levels of aging nerve cells and promote their cellular metabolisms.

Another aspect of the present invention is the discovery of a group of high-energy compounds that can be effective in enhancing the energy levels of cells, particularly the neuronal cells.

Another aspect of the present invention is to identify a high-energy compound(s) that can slow down the aging or death process of a cell, and particularly a neuronal cell.

Another aspect of the present invention is to identify a high-energy compound(s) that can serve as safe, effective, and affordable medication(s) to extend the lifespan of aging or old neuronal cells, thereby delaying the onset of a neurodegenerative disease or disorder, such as the Alzheimer's disease.

Another aspect of the present invention is to identify a high-energy compound(s) that can be used as prototype(s) or precursor(s) for synthesizing chemical derivative(s) of brain-penetrating, or slow- or sustained-release form(s) for use as more efficient and long-lasting medication(s) for a neurodegenerative disease or disorder, such as the Alzheimer's disease.

Another aspect of the present invention is to identify a high-energy compound(s) that can be used to boost the efficacy of conventional medications, which target, for example, free-radicals, toxic proteins, ion imbalances, inflammation, cholesterol, etc.

Another aspect of the present invention is to identify a high-energy compound(s) that can reduce the toxic amyloid protein levels, thereby treating or preventing the Alzheimer's disease.

Another aspect of the present invention is to identify a high-energy compound(s) that slows down the process of cell death induced by energy deficiency or free radicals.

Another aspect of the present invention is to identify a high-energy compound(s) that can be used to treat or prevent Alzheimer's disease, Parkinson's disease, stroke, osteoporosis, muscle atrophy, or other degenerative diseases.

Another aspect of the present invention includes a method of treating or preventing a neurodegenerative disease, which includes administering an effective amount of a compound to a subject in need thereof, wherein the compound comprises a high-energy compound, such as adenosine triphosphate (ATP), guanosine triphosphate (GTP), phosphocreatine (PCr), acetyl coenzyme A (ACoA), phosphoenol pyruvate (PEP), or a combination thereof.

Another aspect of the present invention includes a method of boosting bioenergetics of a cell, which includes subjecting the cell to an effective amount of a compound, such as adenosine triphosphate (ATP), guanosine triphosphate (GTP), phosphocreatine (PCr), acetyl coenzyme A (ACoA), phosphoenol pyruvate (PEP), or a combination thereof.

Another aspect of the present invention includes a method of enhancing survival of a cell, which includes subjecting the cell to an effective amount of a compound, such as adenosine triphosphate (ATP), guanosine triphosphate (GTP), phosphocreatine (PCr), acetyl coenzyme A (ACoA), phosphoenol pyruvate (PEP), or a combination thereof.

### BRIEF DESCRIPTION OF THE DRAWINGS

One of the above and other aspects, novel features and advantages of the present invention will become apparent from the following detailed description of the non-limiting preferred embodiment(s) of invention, illustrated in the accompanying drawings, wherein:

FIGS. 1A-B show that high-energy compounds promoted APP α-processing in a dose-related manner in SH-SY5Y cells;

FIG. 1A shows cultured cells were treated with ATP, phosphocreatine (PCr) and acetyl-coA and their cognate counterparts ADP, Cr and CoA at the indicated concentrations for two hours and conditioned medium was analyzed for sAPPα by Western blotting;

FIG. 1B shows quantitative measurements of the band area and density in FIG. 1A. Values are means±SEM (*p<0.05; **p<0.01; n≧5);

FIGS. 2A-B show relative potencies of several HECs in promoting sAPPα release;

FIG. 2A shows six HECs were tested in comparison with glutamate (Glu) at 1 mM each under identical experimental conditions. In the experiments testing the effects of rotenone (Rot) and NaN₃ (N3), the energy inhibitors were pre-incubated with the cells for 15 minutes prior to the addition of ATP;

FIG. 2B shows EC₅₀ of ATP calculated under the experimental conditions described in Methods. Means±SEM (*p<0.05; **p<0.01; n≧3);

FIGS. 3A-B show HECs offset the inefficient APP α-processing in aged fibroblasts;

FIG. 3A shows western blotting of sAPPα release from five young and five aged human skin fibroblasts. The ages of the individual donors were given on the top or bottom of the bands. Results for ATP (0.5 mM)-treated aged cells were also shown (Old+ATP);

FIG. 3B shows plotting of the scanning data of the bands in FIG. 3A, together with the effects of PEP (0.5 mM) in old cells (Old+PEP). The numbers in the parentheses denote percentile values relative to the young cells. Means±SEM (*p<0.05; **p<0.01; ***p<0.001);

FIG. 4 shows rotenone or H₂O₂ inhibited sAPPα release in the cells and reversal of this effect by HECs. Left, sAPPα release from young cells treated with 0.2 μM rotenone alone (Y+Rot) or co-treated with 1.5 mM ATP or phosphoenol pyruvate (PEP) (Y+Rot+ATP and Y+Rot+PEP). Data for aged cells from FIGS. 3A-B were shown for comparison (Old). Right, the effects of 0.2 mM H₂O₂ alone in the young cell (Y+H₂O₂) or co-treated with 1.5 mM each of ATP or acetyl-coA (ACoA). Means±SEM (*p<0.05; **p<0.01; vs. the respective controls as indicated; n>3);

FIGS. 5A-C show high-energy compounds (HECs) boosted the cell survival in the death process induced by rotenone or H₂O₂;

FIG. 5A shows representative morphologic changes of SH-SY5Y cells before and after treatments with 0.2 μM rotenone alone (Rot) or pre-incubated for 30 min with 1 mM phosphoenol pyruvate (PEP) (Rot+PEP) prior to the addition of rotentone. At the time period indicated, cells were visualized under phase-contrast microscopy (magnification 200×). Arrows denote the preserved neuronal processes;

FIG. 5B shows quantitative measurements of cell viability by MTT assay. Cells treated with 0.2 μM rotenone alone or pre-treated with 1 mM each of the HECs, as indicated; and

FIG. 50 shows cells treated with 0.5 mM H₂O₂ alone or pre-treated with 1 mM each of the selected HECs. Values are means±SEM (*p<0.05; **p<0.01, versus the untreated control; n≧5).

### DETAILED DESCRIPTION OF THE PREFERRED EMBODIMENT(S) OF THE INVENTION

Physiological or α-processing of amyloid-β precursor protein prevents the formation of Aβ, which is deposited in the aging brain and may contribute to Alzheimer's disease. As such, drugs promoting this pathway could be useful for prevention of the disease. Along this line, we searched through a number of substances and unexpectedly found that a group of high-energy compounds (HECs), namely ATP, phosphocreatine and acetyl coenzyme A, potently increased APP α-processing in cultured SH-SY5Y cells, whereas their cognate counterparts, i.e., ADP, creatine or coenzyme A did not show the same effects. Other HECs such as GTP, CTP, phosphoenol pyruvate and S-adenosylmethionine also promoted APP α-processing with varying potencies and the effects were abolished by energy inhibitors rotenone or NaN₃. The overall efficacy of the HECs in the process ranged from three- to four-fold, which was significantly greater than that exhibited by other physiological stimulators such as glutamate and nicotine. This suggested that the HECs were perhaps the most efficient physiological stimulators for APP α-processing in vitro. Moreover, the HECs largely offset the inefficient APP α-processing in aged human fibroblasts or in cells impaired by rotenone or H₂O₂. Most importantly, some HECs markedly boosted the survival rate of SH-SY5Y cells in the death process induced by energy suppression or oxidative stress. These findings suggest a new regulatory mechanism for the putative α-secretase and thus will help substantially in its identification. At the same time, the study raises the possibility that the HECs may be useful to energize and strengthen the aging brain cells to slow down the progression of Alzheimer's disease.

### Materials and Methods

### Materials

Human neuroblastoma SH-SY5Y and human skin fibroblasts from young and aged donors were purchased from ATCC (Gaithersburg, Md., USA). ATP, GTP, CTP, PCr, ACoA, PEP, ADP, GDP, Cr, CoA, PEP, CTP, SAM, glutamate and antibodies to N-terminus of APP (22c11) and β-actin were from EMD Millipore (Temecula, Calif., USA). Precast sodium dodecyl sulfate-polyacrylamide gel electro-phoresis (SDS-PAGE) gels were from Invitrogen (Carlsbad, Calif., USA). NaN₃, rotenone, MTT toxicity assay kit, Dulbecco's modified Eagle's medium (DMEM) and other chemicals were from Sigma-Aldrich (St. Louis, Mo., USA).

### Methods

### Cell Culture and APP Secretion Assays

SH-SY5Y and fibroblast cells were routinely maintained in DMEM supplemented with 10% bovine fetal serum and 100 μg/ml of streptomycine and 100 U/ml of penicillin. For APP secretion assay, the cells were subcultured in the 12-well plate until confluence and then pre-incubated in the serum-free DMEM for 1 hour before the addition of the testing agents. After incubation for 2 hours at 37° C., conditioned medium was collected and cleared by centrifugation. Proteins were precipitated by trichloroacetic acid and processed for gel electrophoresis and Western blotting as described previously (Weidemann et al. 1989; Chen and Fernandez, 2004). A near-infrared dye-conjugated secondary antibody (LI-COR Biosci., NE, USA) was used and visualized with a LI-COR Odyssey Infrared Imaging System.

### Cell Survival and Viability MTT Assay

SH-SY5Y cells were subcultured in the 12-well plate in DMEM containing 10% fetal bovine serum until 50% confluency. For experiment, the medium was replaced with serum-free DMEM and testing agents were added. The culture was continued in the incubator and parallel samples were taken out at various time intervals for microscopic examination or cell viability assay using an MTT toxicity assay kit (Sigma, Mo.) according to the manufacturer's instructions with modifications (Chen and Fernandez, 2004).

### Statistical Analysis

Evaluation of the differences in fold increase of APP secretion was carried out using the computer-assisted paired Student t-test and cell viability was calculated using ANOVA with post-hoc test.

### Results

### HECs Promote APP α-Processing

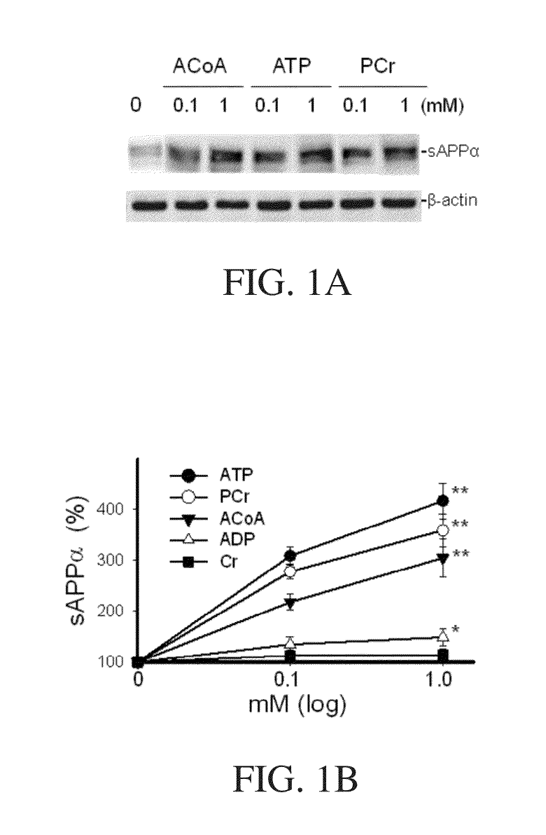

To find effective compounds that may benefit the aging brain, we tested a number of substances by measuring their effectiveness in promoting the release of the secreted form of APP by α-processing (sAPPα) from cultured SH-SY5Y neurons. The tested substances included current or experimental drugs, herbal extracts, nutritional supplements and other compounds. These experiments revealed that among the substances, ATP was highly effective in promoting sAPPα release (FIG. 1). As APP α-processing has been found to be sensitive to energy perturbations (Gabuzda et al. 1994; Webster et al., 1998; Hoyer et al. 2005), the finding prompted us to speculate that the observed effects of ATP could be attributable to its high energy content. Subsequent experiments confirmed this speculation.

It was found that, similar to ATP, two other HECs, acetyl-coA and PCr also exhibited strong and dose-related effects on APP α-processing. As shown, these three HECs at 1 mM each promoted sAPPα release by 3.3-, 3.8- and 4.2-fold relative to basal line, respectively (FIGS. 1A and 1B). The presence of high-energy bonds in these compounds was necessary for the observed effects as their cognate counterparts without the same energy content, namely adenosine diphosphate (ADP), creatine (Cr) or coenzyme A (CoA), did not show the same effects (FIG. 1B, CoA not shown). Of note was that acetyl-coA possesses an ˜acetyl (thioester) energy-rich bond (Su and Abumrad, 2009).

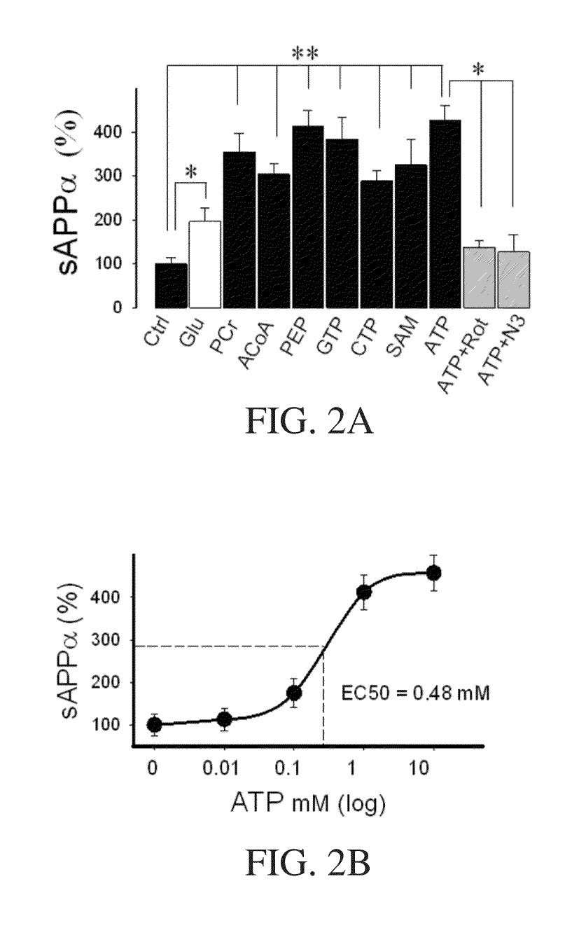

Further studies showed that several other HECs, namely PEP, GTP, CTP and SAM (1 mM each), also exerted similar effects on APP secretion (FIG. 2A). Overall, they displayed varying magnitudes of efficacy that ranged from three- to more than four-fold when tested at the same concentration. ATP seemed to be the most efficient one with an EC₅₀=0.48 mM (FIG. 2B). Such effectiveness was significantly greater than that of glutamate (2.3-fold) (FIG. 2A), nicotine or bradykinin (not shown).

Nevertheless, their stimulatory effects were completely abolished when these HECs were tested in the presence of respiratory chain inhibitor rotenone (20 μM), or cytochrome oxidase inhibitor NaN₃ (25 mM) (FIG. 2A, showing their effects on ATP only, the effects on other HECs not shown). Similar effects on APP secretion were also observed when ATP, GTP and PCr were tested in cultured human kidney cell HEK293 (not shown) or human skin fibroblasts (see below). Thus, the observed effects of the HECs were presumably a general phenomenon in various cell types.

### HECs Offset the Effects of Age-Related Impairments

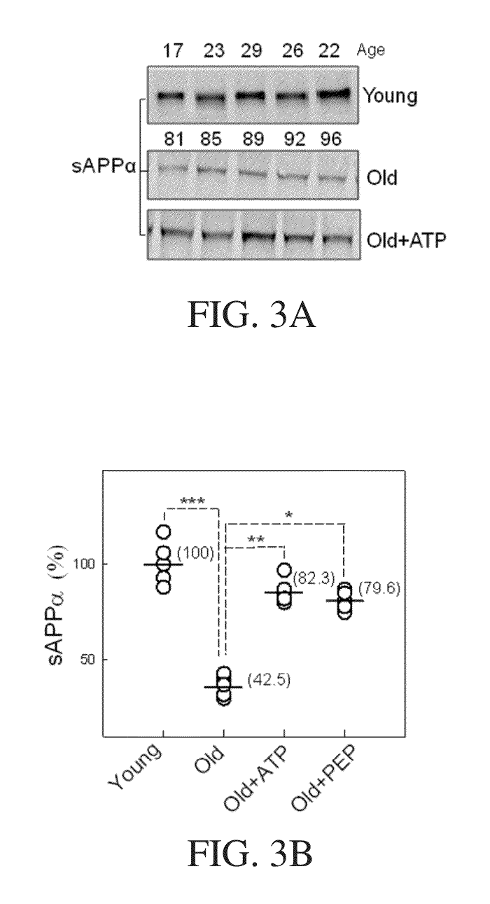

Mitochondrial function and the HECs levels are decreased in the aging brain and AD (Owen and Sunram-Lea, 2011; Foy et al. 2011; Meier-Ruge and Bertoni-Freddari, 1999) and the resulting energy crisis underlies various age-related changes including decreased APP α-processing (Lannfelt et al., 1995; Kern et al., 2006). We compared sAPPα release in human skin fibroblast lines obtained from five young (age 17-29) and five aged donors (age 81-96). It was found that the average level of sAPPα release from aged cells decreased to 42.5% of the young cell control in the resting state (FIGS. 3A and 3B). Interestingly, ATP or PCr (0.5 mM each) enhanced the sAPPα release in aged cells to 82.3% and 79.6% of the young cell level, respectively (FIGS. 3A and 3B; effects of PCr not shown in 3A).

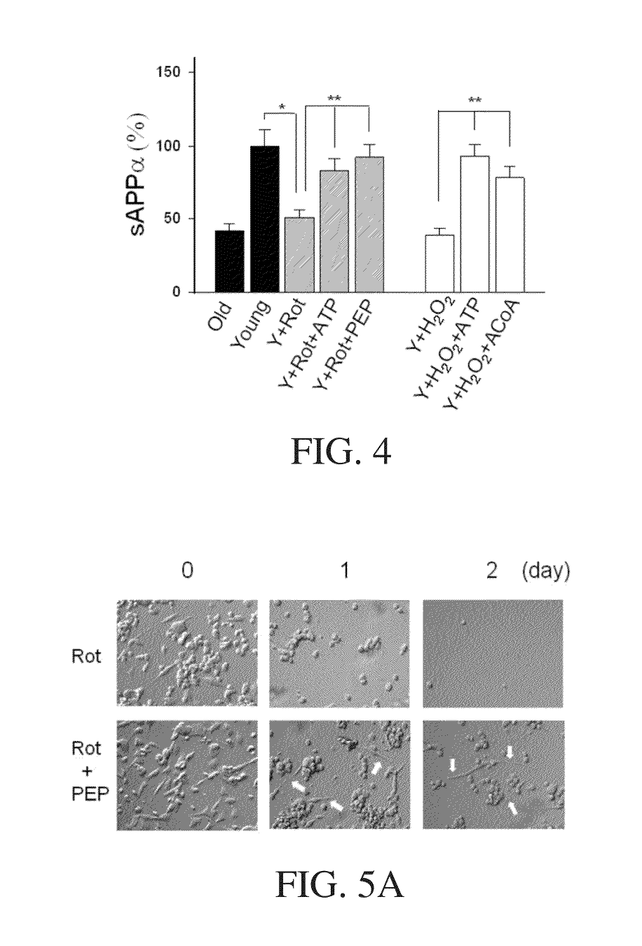

To see whether the inefficient APP α-processing in aged cells could be linked to energy suppression, we tested the effects of rotenone in the young cells. As shown, treating young cells with 0.2 μM rotenone for two hours reduced the sAPPα level to 52.3% of the control. And importantly, this reduced level was largely restored by co-treatment of 1.5 mM each of ATP or PEP, which brought the sAPPα level back to 79.2% and 90.3% of the control, respectively (FIG. 4, left panel).

Dysfunctional mitochondria and energy depletion in the aging brain occur with concomitant free radical accumulation (Bonda et al. 2009). We found that treating young cells with 0.2 mM H₂O₂ for two hours decreased sAPPα release to 46.1% of the young cell control and, again, this effect of H₂O₂ was largely attenuated by co-treatment of the cells with 1.5 mM each of ATP or ACoA, which increased the sAPPα level to 89.3 and 74.5% of the control, respectively (FIG. 4, right panel).

### HECs Promote Cell Survival

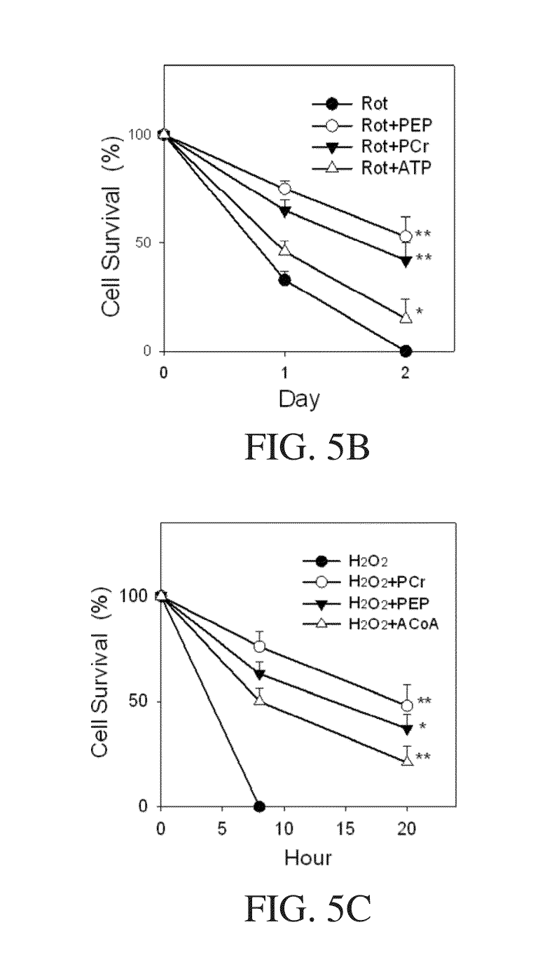

If the HECs offset age-related impairments to APP processing, it would be possible that the HECs also slowdown the cell death induced by the same impairments. This possibility was examined in the death process of SH-SY5Y cells induced by rotenone, an agent that causes neuronal death in cultured cells and animal models for AD and Parkinson's disease (Chaves et al. 2010; Hoglinger et al. 2005). In our hands, 0.2 μM rotenone caused a progressive neuronal death with a concomitant cell shrinkage and disappearance of the neuronal processes, as visualized under microscopy (FIG. 5A, upper panel). However, pre-incubation of the cells with PEP (1 mM) for 30 min prior to the addition of rotenone substantially preserved the cell shape and the processes (FIG. 5A, lower panel). A quantitative cell viability assay showed that rotenone alone caused all cells to die by 2 days, but pre-incubation of PEP, PCr or ATP (1 mM each) enhanced the cell survival to 53%, 42% or 15% of the untreated control, respectively (FIG. 5B). Meanwhile, acetyl-coA and GTP (1 mM each) also increased cell survival to 35% and 17% under the same experimental conditions (not shown).

Finally, we tested the effects of the HECs on cell death induced by H₂O₂ and found that while 0.6 mM H₂O₂ killed all cells by 10 hours, pre-treatment with 1 mM each of PCr, PEP or acetyl-coA considerably increased the cell survival to 49%, 38% and 21% of the control, respectively, by 20 hours (FIG. 5C). At the same time, ATP or GTP also moderately increased cell survival (not shown).

Thus, these HECs displayed significant neuroprotective effects in cultured cells, though with varying degrees of efficacy. Overall, it appeared that PCr, PEP or acetyl-coA were more efficient than ATP and GTP under the experimental conditions. Although the reasons were unclear, it is possible that this may relate to the energy levels they released, durability of the actions in the cell or compatibility with the specific cell type.

### Discussion

### Free Energy in APP Processing

In this study we found that HECs promote sAPPα secretion by three- to four-fold, an efficacy that is greater than that reported for other physiological stimulators, such as glutamate, nicotine, bradykinin and growth factors (averaged two- to three-fold) (Jolly-Tornetta et al. 1998; Kim et al. 1997; Mills et al. 1997; Solano et al. 2000; Fisher et al. 2003). It thus appears that these HECs are perhaps the most efficient physiological stimulators for the APP secretory pathway when tested in vitro, though perhaps less efficient than phorbol esters (three- to six-fold reported in various cell types) (Efthimiopoulos, et al. 1994; Bauxbum et al. 1990).

The HECs may exert their observed actions through diverse pathways. For example, ATP and GTP may interact with a family of purinergic receptors (P2Y) and activate the G protein-related signal transduction cascades (Abbracchio et al. 2009). PCr and Cr are taken up into cell by a sodium-dependent transporter and serve as a primary energy reserve system in the body (Adhihetty and Beal, 2008). Acetyl-CoA can directly enter mitochondria and affects oxidative phosphorylation (Su and Abumrad, 2009). However, despite the multiple initial paths, our data suggest that the free energy they released appears to be the converging point of these pathways and is responsible for the observed effects on APP α-processing. Consistent with this view, other energy-rich compounds have been reported to exhibit similar effects (Camden et al., 2005; Epis et al., 2008).

APP α-processing is sensitively regulated by a myriad of metabolic or signal-transduction pathways (Lichtenthaler, 2011; review) and is also sensitive to energy alterations (Gabuzda et al. 1994; Webster et al., 1998; Hoyer et al. 2005). Now our findings in this study will make the regulatory mechanism of APP α-processing or α-secretase an even more attractive subject for study. By highlighting free energy as an additional regulator, our study has revealed an intriguing, energy-dependent feature of α-secretase. Future studies on this “signature trait” may lead to a better understanding of the true identity of this key enzyme and its central regulatory role in the overall APP metabolism in brain aging and AD (Chen and Fernandez, 2004; Nguyen et al., 2012).

### HECs Protect Cells Against Age-Related Impairments

Our findings that the impairments of energy suppression and oxidative stress on APP α-processing can be partially attenuated by HECs may suggest a promising approach to reduce Aβ production in the aging brain, as the two processing pathways compete for the same APP pool (Skovronsky et al. 2000; Frautschy et al. 1998; Chen and Fernandez, 2004). More importantly, that the HECs substantially rescue the cells from death process invoked by energy inhibition or oxidative stress further suggest that the HECs can enhance the cell resilience against age-related impairments. Thus, these compounds may serve as potential medications for intervention in brain aging and AD.

The precise mechanism underlying the protective effects of the HECs is unclear at present, but it is possible that increased sAPPα or decreased Aβ may each contribute a part. Although this issue may be clarified by using APP mutant cells in future studies, the use of natural cells is this study is more relevant to brain aging and early phase of sporadic AD. Our data suggest that boosting cell bioenergetics is a pertinent mechanism in the protective effects of the HECs. This view is also based on following considerations.

Brain function is highly energy-dependent and enhancing energy metabolisms can facilitate brain oxygenation, circuit integration and antioxidant defense (Bonda et al. 2009; McDaniel et al. 2003). Indeed, while the overall mechanisms of AD have remained highly controversial today, there is a broad consensus suggesting that energy depletion or mitochondrial dysfunction is perhaps the earliest measurable defect in aging, which underlies a wide range of downstream impairments such as oxidative stress, protein aggregations and ion dyshomeostasis (Castellani et al. 2002; Blass, 2000; Beal, 2008; Swerdlow, 2011).

This energy-centric hypothesis has been substantially supported by an increasing number of studies showing that energy suppression triggers Aβ overproduction and tau aggregation in cultured cells (Hoyer et al. 2005; Chaves et al. 2010; Leuner et al., 2012), causes Aβ deposition and tauopathy in the animal brain (Leuner et al. 2012; Grünblatt et al. 2007; Höglinger, 2005), and also directly impairs animal learning and memory (Lannert and Hoyer, 1998; Luques et al. 2007). These findings can explain an important empirical knowledge that physical and brain exercises should serve as the first line of defense against age-related cognitive deterioration in the elderly population.

### Potential Use of HECs for AD Prevention

Moreover, given the key role of energy crisis in brain aging, it is conceivable that boosting energy levels would be an ideal point of entry for pharmaceutical intervention in AD. In this regard, our findings may provide a promising basis for future studies. As many HECs in the body have not been fully characterized, it is anticipated that future studies will discover more efficient and brain-specific HECs, or develop their longer-lasting derivatives to serve as safe, effective and cost-economic medications for AD. In this regard, several energy-rich supplements have been found to exert some neuroprotective effects (Owen and Sunram-Lea, 2011; Adhihetty and Beal, 2008; Tchantchou et al. 2008; Montgomery et al. 2003). It is likely that their long-term and early use in the aging population, enhanced by healthy lifestyle, will result in more prominent effects.

High-energy compounds (HECs) may also improve the efficacy of current or experimental drugs that target neurotransmitters, Aβ or tau aggregates, free radicals, ion imbalances, inflammation and other lesions (Salomone et al. 2012; Herrmann et al. 2011; reviews). These drugs have not shown prominent results in the clinic today, but by improving cellular bioenergetics, they may display better effects for AD, a multifactorial disorder that requires multifaceted intervention.

Finally, HECs may also benefit other late-life degenerative conditions such as Parkinson's, stroke, osteoporosis and muscle dystrophy in which energy depletion is also the underpinning factor. As some HECs have been used to build muscles and bones, it is possible that “strengthen the brain like muscle and bone” may emerge as a useful strategic thinking in our endeavor to energize and protect aging brains, thereby extending their lifespan to catch up with the extended life expectancy of the aging population.

The invention also provides pharmaceutical or dietary supplemental compositions comprising one or more high-energy compounds (HECs) disclosed herein. Accordingly, the compound(s), can be formulated for oral or parenteral administration for the therapeutic or prophylactic treatment of neurodegenerative diseases or conditions, particularly the Alzheimer's disease.

By way of illustration, the compound(s) can be admixed with conventional pharmaceutical carriers and/or excipients and used in the form of tablets, capsules, elixirs, suspensions, syrups, wafers, and the like. Such pharmaceutical compositions may contain from about 0.1 to about 90% by weight of the active HEC compound(s), and more generally from about 10 to about 30%. The pharmaceutical compositions may contain common carriers and excipients, such as corn starch, gelatin, lactose, sucrose, microcrystalline cellulose, kaolin, mannitol, dicalcium phosphate, sodium chloride, and alginic acid. Disintegrators commonly used in the formulations of this invention include croscarmellose, microcrystalline cellulose, corn starch, sodium starch glycolate and alginic acid.

A liquid composition will generally consist of a suspension or solution of the compound or pharmaceutically acceptable salt in a suitable liquid carrier(s), for example ethanol, glycerine, sorbitol, non-aqueous solvent such as polyethylene glycol, oils or water, optionally with a suspending agent, a solubilizing agent (such as a cyclodextrin), preservative, surfactant, wetting agent, flavoring or coloring agent.

Alternatively, a liquid formulation can be prepared from a reconstitutable powder. For example, a powder containing active compound, suspending agent, sucrose and a sweetener can be reconstituted with water to form a suspension; and a syrup can be prepared from a powder containing active ingredient, sucrose and a sweetener.

A composition in the form of a tablet can be prepared using any suitable pharmaceutical carrier(s) routinely used for preparing solid compositions. Examples of such carriers include magnesium stearate, starch, lactose, sucrose, microcrystalline cellulose and binders, for example polyvinylpyrrolidone. The tablet can also be provided with a color film coating, or color included as part of the carrier(s). In addition, active compound can be formulated in a controlled release dosage form as a tablet comprising a hydrophilic or hydrophobic matrix.

A composition in the form of a capsule can be prepared using routine encapsulation procedures, for example, by incorporation of active compound and excipients into a hard gelatin capsule. Alternatively, a semi-solid matrix of active compound and high molecular weight polyethylene glycol can be prepared and filled into a hard gelatin capsule; or a solution of active compound in polyethylene glycol or a suspension in edible oil, for example liquid paraffin or fractionated coconut oil can be prepared and filled into a soft gelatin capsule.

Tablet binders that can be included are acacia, methylcellulose, sodium carboxymethylcellulose, poly-vinylpyrrolidone (Povidone), hydroxypropyl methylcellulose, sucrose, starch and ethylcellulose. Lubricants that can be used include magnesium stearate or other metallic stearates, stearic acid, silicone fluid, talc, waxes, oils and colloidal silica.

Flavoring agents such as peppermint, oil of wintergreen, cherry flavoring or the like can also be used. Additionally, it may be desirable to add a coloring agent to make the dosage form more attractive in appearance or to help identify the product.

The compounds of the invention and their pharmaceutically acceptable salts that are active when given parenterally can be formulated for intramuscular, intrathecal, or intravenous administration. A typical composition for intramuscular or intrathecal administration consists of a suspension or solution of active ingredient in an oil, for example arachis oil or sesame oil. A typical composition for intravenous or intrathecal administration consists of a sterile isotonic aqueous solution containing, for example active ingredient and dextrose or sodium chloride, or a mixture of dextrose and sodium chloride. Other examples of aqueous solution are lactated Ringers injection, lactated Ringer's plus dextrose injection, Normosol-M and dextrose, Isolyte E, acylated Ringer's injection, and the like. Optionally, a co-solvent, for example, polyethylene glycol; a chelating agent, for example, ethylenediamine tetracetic acid; a solubilizing agent, for example, a cyclodextrin; and an anti-oxidant, for example, sodium metabisulphite, may be included in the formulation. Alternatively, the solution can be freeze dried and then reconstituted with a suitable solvent just prior to administration.

The compound of the invention which are active on rectal administration can be formulated as suppositories. A typical suppository formulation will generally consist of active ingredient with a binding and/or lubricating agent such as a gelatin or cocoa butter or other low melting vegetable or synthetic wax or fat.

The active compound is effective over a wide dosage range and is generally administered in a therapeutically effective amount. It, will be understood, however, that the amount of the compound actually administered will be determined by a physician, in the light of the relevant circumstances, including the condition to be treated, the chosen route of administration, the actual compound administered and its relative activity, the age, weight, and response of the individual patient, the severity of the patient's symptoms, and the like. Suitable doses are selected to effect a blood concentration of about 100-300 μM, preferably 100 μM.

According to the invention, a compound can be administered in a single daily dose or in multiple doses per day. The treatment regimen may require administration over extended periods of time, for example, for several days, for from one to six weeks, or longer.

Suitable formulations for use in the present invention can be found in Remington's Pharmaceutical Sciences, Mace Publishing Company, Philadelphia, Pa., 17th ed. (1985).

While this invention has been described as having preferred sequences, ranges, steps, order of steps, materials, structures, symbols, indicia, graphics, color scheme(s), shapes, configurations, features, components, or designs, it is understood that it is capable of further modifications, uses and/or adaptations of the invention following in general the principle of the invention, and including such departures from the present disclosure as those come within the known or customary practice in the art to which the invention pertains, and as may be applied to the central features hereinbefore set forth, and fall within the scope of the invention and of the limits of the claims appended hereto or presented later. The invention, therefore, is not limited to the preferred embodiment(s) shown/described herein.

### REFERENCES

The following references, and any cited in the disclosure herein, are hereby incorporated herein in their entirety by reference.
1. Abbracchio M. P., Burnstock G., Verkhratsky A. and Zimmermann H. (2009) Purinergic signalling in the nervous system: an overview. Trends Neurosci. 32, 19-29.
2. Adhihetty P. J. and Beal M. F. (2008) Creatine and its potential therapeutic value for targeting cellular energy impairment in neurodegenerative diseases. Neuromolecular Med. 10, 275-290.
3. Beal M. F. (1998) Mitochondrial dysfunction in neurodegenerative diseases. Biochim Biophys Acta. 1366, 211-223.
4. Blass J. P. (2000) The mitochondrial spiral. An adequate cause of dementia in the Alzheimer's syndrome. Ann NY Acad Sci. 924, 170-183.
5. Bonda D. J., Wang X., Gustaw-Rothenberg K. A., Perry G., Smith M. A. and Zhu X. (2009) Mitochondrial Drugs for Alzheimer Disease. Pharmaceuticals (Basel). 2, 287-298.
6. Buxbaum J. D., Gandy S. E., Cicchetti P., Ehrlich M. E., Czernik A. J., Fracasso R. P., Ramabhadran T. V., Unterbeck A. J. and Greengard P. (1990) Processing of Alzheimer beta/A4 amyloid precursor protein: modulation by agents that regulate protein phosphorylation. Proc Natl Acad Sci USA. 87, 6003-6006.
7. Camden J. M., Schrader A. M., Camden R. E., González F. A. et al. (2005) P2Y2 nucleotide receptors enhance alpha-secretase-dependent amyloid precursor protein processing. J Biol Chem. 280, 18696-18702.
8. Castellani R., Hirai K., Aliev G., Drew K. L., Nunomura A. et al. (2002) Role of mitochondrial dysfunction in Alzheimer's disease. J Neurosci Res. 70, 357-360.
9. Chaves R. S., Melo T. Q., Martins S. A. and Ferrari M. F. (2010) Protein aggregation containing β-amyloid, α-synuclein and hyperphosphorylated tau in cultured cells of hippocampus, substantia nigra and locus coeruleus after rotenone exposure. BMC Neurosci. 11, 144.
10. Chen M. and Fernandez H. L. (2004) Stimulation of beta-amyloid precursor protein alpha-processing by phorbol ester involves calcium and calpain activation. Biochem. Biophys. Res. Commun. 316, 332-340.
11. Epis R., Marcello E., Gardoni F., Longhi A., Calvani M., lannuccelli M. et al. (2008) Modulatory effect of acetyl-L-carnitine on amyloid precursor protein metabolism in hippocampal neurons. Eur J. Pharmacol. 597, 51-56.
12. Esch F. S., Keim P. S., Beattie E. C., Blacher R. W. Culwell A. R. et al. (1990) Cleavage of amyloid beta peptide during constitutive processing of its precursor. Science 248, 1122-1124.
13. Efthimiopoulos S, Felsenstein K M, Sambamurti K, Robakis N K, Refolo L M. (1994) Study of the phorbol ester effect on Alzheimer amyloid precursor processing: sequence requirements and involvement of a cholera toxin sensitive protein. J Neurosci Res. 38, 81-90.
14. Etcheberrigaray R., Tan M., Dewachter I., Kuiperi C. and Van Der Auwera I. et al. (2004) Therapeutic effects of PKC activators in Alzheimer's disease transgenic mice. Proc Natl Acad Sci USA. 101, 11141-11146.
15. Fisher A., Pittel Z., Haring R., Bar-Ner N., Kliger-Spatz M. et al. (2003) M1 muscarinic agonists can modulate some of the hallmarks in Alzheimer's disease: implications in future therapy. J Mol Neurosci. 20, 349-356.
16. Foy C. M., Daly E. M., Glover A., O'Gorman R., Simmons A. et al. (2011) Hippocampal proton MR spectroscopy in early Alzheimer's disease and mild cognitive impairment. Brain Topogr. 24, 316-322. (Pcr lower)
17. Frautschy S. A., Horn D. L., Sigel J. J., Harris-White M. E., Mendoza J. J. et al. (1998) Protease inhibitor coinfusion with amyloid beta-protein results in enhanced deposition and toxicity in rat brain. J. Neurosci. 18, 8311-8321.
18. Gabuzda D., Busciglio J., Chen L. B., Matsudaira P. and Yankner B. A. (1994) Inhibition of energy metabolism alters the processing of amyloid precursor protein and induces a potentially amyloidogenic derivative. J Biol Chem. 269, 13623-13628.
19. Grünblatt E., Salkovic-Petrisic M., Osmanovic J., Riederer P. and Hoyer S. (2007) Brain insulin system dysfunction in streptozotocin intracerebroventricularly treated rats generates hyperphosphorylated tau protein. J. Neurochem. 101, 757-770.
20. Herrmann N., Chau S. A., Kircanski I. and Lanctôt K L. (2011) Current and emerging drug treatment options for Alzheimer's disease: a systematic review. Drugs. 71, 2031-2065.
21. Höglinger G. U, Lannuzel A., Khondiker M. E., Michel P. P., Duyckaerts C. et al. (2005) The mitochondrial complex I inhibitor rotenone triggers a cerebral tauopathy. J Neurochem. 95, 930-939.
22. Hoyer A, Bardenheuer H. J., Martin E., Plaschke K. (2005) Amyloid precursor protein (APP) and its derivatives change after cellular energy depletion. An in vitro-study. J Neural Transm. 112, 239-253.
23. Jolly-Tornetta C., Gao Z. Y., Lee V. M., Wolf B. A. (1998) Regulation of amyloid precursor protein secretion by glutamate receptors in human Ntera 2 neurons. J Biol Chem. 273, 14015-14021.
24. Kern A., Roempp B., Prager K., Walter J., Behl C. (2006) Down-regulation of endogenous amyloid precursor protein processing due to cellular aging. J Biol Chem. 281, 2405-2413.
25. Kim S. H., Kim Y. K., Jeong S. J. et al. (1997) Enhanced release of secreted form of Alzheimer's amyloid precursor protein from PC12 cells by nicotine. Mol Pharmacol. 52, 430-436.
26. Lannert H. and Hoyer S. (1998) Intracerebroventricular administration of streptozotocin causes long-term diminutions in learning and memory abilities and in cerebral energy metabolism in adult rats. Behav Neurosci. 112, 1199-1208.
27. Lannfelt L., Basun H., Wahlund L.-O., Rowe B. A. and Wagner S. L. (1995) Decreased alpha-secretase-cleaved amyloid precursor protein as a diagnostic marker for Alzheimer's disease. Nature Med. 1, 829-832.
28. Lehninger A. L. (1975) Biochemistry. 2nd Edn. Worth Publishing. New York.
29. Leuner K., Schütt T., Kurz C., Eckert S. H., Schiller C. et al. (2012) Mitochondrion-derived reactive oxygen species lead to enhanced amyloid beta formation. Antioxid Redox Signal. 16, 1421-1433.
30. Lichtenthaler S. F. (2011) Alpha-secretase in Alzheimer's disease: molecular identity, regulation and therapeutic potential. J Neurochem. 116, 10-21.
31. Luques L., Shoham S. and Weinstock M. (2007) Chronic brain cytochrome oxidase inhibition selectively alters hippocampal cholinergic innervation and impairs memory: prevention by ladostigil. Exp Neurol. 206, 209-219.
32. McDaniel M. A., Maier S. F. and Einstein G. O. (2003) “Brain-specific” nutrients: a memory cure? Nutrition 19, 957-975.
33. Meier-Ruge W. A. and Bertoni-Freddari C. (1999) Mitochondrial genome lesions in the pathogenesis of sporadic Alzheimer's disease. Gerontology 45, 289-297. (ACoA)
34. Mills J., Laurent Charest D., Lam F., Beyreuther K., Ida N., Pelech S. L. and Reiner P. B. (1997) Regulation of amyloid precursor protein catabolism involves the mitogen-activated protein kinase signal transduction pathway. J Neurosci. 17, 9415-9422.
35. Montgomery S. A., Thal L. J. and Amrein R. (2003) Meta-analysis of double blind randomized controlled clinical trials of acetyl-L-carnitine versus placebo in the treatment of mild cognitive impairment and mild Alzheimer's disease. Int Clin Psychopharmacol. 18, 61-71.
36. Nguyen H. T., Sawmiller D. R., Wu Q., Maleski J. J. and Chen M. (2012) Evidence supporting the role of calpain in the α-processing of amyloid-β precursor protein. Biochem Biophys Res Commun. 420, 530-536.
37. Nicholls D. G. and Ferguson S. J. (2002) Bioenergetics 3^{rd} Edn. Academic Press, London.
38. Owen L. and Sunram-Lea S. I. (2011) Metabolic agents that enhance ATP can improve cognitive functioning: a review of the evidence for glucose, oxygen, pyruvate, creatine, and L-carnitine. Nutrients 3, 735-755.
39. Salomone S., Caraci F., Leggio G. M., Fedotova J. and Drago F. (2012) New pharmacological strategies for treatment of Alzheimer's disease: focus on disease modifying drugs. Br J Clin Pharmacol. 73, 504-517.
40. Skovronsky D. M., Moore D. B., Milla M. E., Doms R. W. and Lee V. M. (2000) Protein kinase C-dependent alpha-secretase competes with beta-secretase for cleavage of amyloid-beta precursor protein in the trans-Golgi network. J. Biol. Chem. 275, 2568-2575.
41. Solano D. C, Sironi M., Bonfini C., Solerte S. B., Govoni S. and Racchi M. (2000) Insulin regulates soluble amyloid precursor protein release via phosphatidyl inositol 3 kinase-dependent pathway. FASEB J. 14, 1015-1022.
42. Su, X. and Abumrad, N. A. (2009) Cellular fatty acid uptake: a pathway under construction. Trends Endocrinol. Metab. 20, 72-77.
43. Sun M. K. and Alkon D. L. (2010) Pharmacology of protein kinase C activators: cognition-enhancing and antidementic therapeutics. Pharmacol Ther. 127, 66-77.
44. Swerdlow R. N. (2011) Brain aging, Alzheimer's disease, and mitochondria. Biochim Biophys Acta 1812, 1630-1639.
45. Tchantchou F, Graves M, Falcone D, Shea T B. (2008) S-adenosylmethionine mediates glutathione efficacy by increasing glutathione S-transferase activity: implications for S-adenosyl methionine as a neuroprotective dietary supplement. J Alzheimers Dis. 14, 323-328.
46. Van Nostrand W. E., Wagner S. L., Shankle W. R. et al. (1992) Decreased levels of soluble amyloid beta-protein precursor in cerebrospinal fluid of live Alzheimer disease patients. Proc Natl Acad Sci USA. 9, 2551-2555.
47. Walsh D. M. and Selkoe D. J. (2004) Deciphering the molecular basis of memory failure in Alzheimer's disease. Neuron 44, 181-193.
48. Webster M. T., Pearce B. R., Bowen D. M. and Francis P T. (1998) The effects of perturbed energy metabolism on the processing of amyloid precursor protein in PC12 cells. J Neural Transm. 105, 839-853.
49. Weidemann A, Konig G, Bunke D, Fischer P, Salbaum J M, Masters C L, Beyreuther K. (1989) Identification, biogenesis, and localization of precursors of Alzheimer's disease A4 amyloid protein. Cell 57, 115-126.
50. Sawmiller D R, Nguyen H T, Markov O, Chen M. (2012) High-energy compounds promote physiological processing of Alzheimer's amyloid-6 precursor protein and boost cell survival in culture. J. Neurochem. (2012) 123, 525-531. Published Online Sep. 21, 2012.

What is claimed is:

1. A method of treating or preventing a neurodegenerative disease, comprising:
a) administering an effective amount of a compound to a subject in need thereof; and
b) wherein the compound comprises a high-energy compound selected from the group consisting of adenosine triphosphate (ATP), guanosine triphosphate (GTP), phosphocreatine (PCr), acetyl coenzyme A (ACoA), phosphoenol pyruvate (PEP), and a combination thereof.

2. The method of claim 1, wherein:
the neurodegenerative disease comprises Alzheimer's disease.

3. The method of claim 2, wherein:
the high-energy compound comprises ATP.

4. The method of claim 1, wherein:
the neurodegenerative disease comprises Parkinson's disease.

5. The method of claim 1, wherein:
the neurodegenerative disease comprises stroke.

6. The method of claim 1, wherein:
the compound is administered orally, intravenously, or subcutaneously.

7. A method of boosting bioenergetics of a cell, comprising:
a) subjecting the cell to an effective amount of a compound; and
b) wherein the compound comprises a high-energy compound selected from the group consisting of adenosine triphosphate (ATP), guanosine triphosphate (GTP), phosphocreatine (PCr), acetyl coenzyme A (ACoA), phosphoenol pyruvate (PEP), and a combination thereof.

8. The method of claim 7, wherein:
the cell comprises a neuronal cell.

9. The method of claim 7, wherein:
the cell comprises a brain cell.

10. The method of claim 7, wherein:
the cell comprises a human neuroblastoma or skin fibroblast.

11. The method of claim 7, wherein:
the cell comprises an aging brain cell.

12. The method of claim 11, wherein:
the compound comprises ATP.

13. A method of enhancing survival of a cell, comprising:
a) subjecting the cell to an effective amount of a compound; and
b) wherein the compound comprises a high-energy compound selected from the group consisting of adenosine triphosphate (ATP), guanosine triphosphate (GTP), phosphocreatine (PCr), acetyl coenzyme A (ACoA), phosphoenol pyruvate (PEP), and a combination thereof.

14. The method of claim 13, wherein:
the cell comprises a neuronal cell.

15. The method of claim 13, wherein:
the cell comprises a brain cell.

16. The method of claim 13, wherein:
the cell comprises a human neuroblastoma or skin fibroblast.

17. The method of claim 13, wherein:
the cell comprises an aging brain cell.

18. The method of claim 17, wherein:
the compound comprises phosphoenol pyruvate (PEP).

19. The method of claim 17, wherein:
the compound comprises phosphocreatine (PCr).

20. The method of claim 17, wherein:
the compound comprises acetyl coenzyme A (ACoA).
